(19) United States
(12) Patent Application Publication
(10) Pub. No.: US 2007/0027443 A1
(43) Pub. Date: Feb. 1, 2007

(54) HAND PIECE FOR THE DELIVERY OF LIGHT AND SYSTEM EMPLOYING THE HAND PIECE

(75) Inventors: Andreas Rose, Sammamish, WA (US); Guenter Herr, Ehringshaushen (DE); Nicolas G. Loebel, Redmond, WA (US)

Correspondence Address: DOBRUSIN & THENNISCH PC, 29 W LAWRENCE ST, SUITE 210, PONTIAC, MI, 48326, US

(73) Assignee: Ondine International, Ltd.

(21) Appl. No.: 11/425,548

(22) Filed: Jun. 21, 2006

Related U.S. Application Data
(60) Provisional application No. 60/694,952, filed on Jun. 29, 2005.

(51) Int. Cl. A61B 18/18; A61C 1/00; A61C 3/00

(52) U.S. Cl. 606/16; 433/29; 606/13

(57) ABSTRACT

The invention described here is an improved hand piece for the delivery of light and a system employing the hand piece. The hand piece typically includes a body and an optical element such as an optical fiber coextensive with the body. The system can include a remote light source and an optical element (e.g., a source optical fiber) for providing light to the hand piece.

### CLAIM OF BENEFIT OF FILING DATE

This application claims the benefit of the filing date of U.S. Provisional Application Ser. No. 60/694,952 filed on Jun. 29, 2005, and incorporated herein by reference for all purposes.

### TECHNICAL FIELD

The present invention generally relates to medical devices. More particularly, the invention relates to a hand piece and system for delivering light typically for medical applications.

### BACKGROUND OF THE INVENTION

Optical fibers have been advantageously used to deliver light in a great multitude of applications. More recently, optical fibers have been employed to deliver light for use in medical applications such as photodynamic therapy (PDT), photodynamic disinfection (PDD), photo-assisted tissue welding or the like. For certain medical applications, it is desirable for an individual to be able to use a hand piece for assisting in delivering light using an optical fiber. However, conventional hand pieces have exhibited various undesirable characteristics and problems. As one example, optical fibers of the hand pieces can be damaged by exposure to certain ambient conditions (e.g., elevated temperatures, humidity or the like such as might be experienced in an autoclave). As another example, such hand pieces can be quite expensive, As yet another example, such hand pieces can exhibit substantial light loss. Accordingly, the present invention provides a hand piece, a system employing the hand piece or both that minimize and/or overcome undesirable characteristics and/or problems exhibited by conventional hand pieces as mentioned above or as will become clear to the skilled artisan from the description below.

### SUMMARY OF THE INVENTION

The present invention is a hand piece used to deliver light in medical applications and possibly other applications as well. A proximal end of the hand piece is typically configured for receiving light from an optical source fiber that delivers light from a remote light source/receiver instrument. Light can be transmitted from the source fiber through an optical fiber of the hand piece to distal end of the hand piece. The distal end can be configured for receiving a removable tip used to delivers light to and/or receives light from an intended application site such as biological tissue of a human or other organism.

The hand piece can be designed to have a unique, modular character that allows the hand piece to be sterilized in an autoclave and allows optical surfaces to be cleaned. The hand piece typically includes a body that can have an ergonomic design and may be considered as part of a central shaft assembly of the hand piece or it can be a separate component or it can be part of a tip. The hand piece can be configured to include a retaining sleeve, which can assist in holding the body onto the rest of the shaft assembly. A retaining nut can also be included as part of the hand piece and it can be configured allow a removable source fiber ferrule to be securely interfaced with the hand piece. When a removable source fiber ferrule is used, then the retaining sleeve and an internal adapter can be employed to work in conjunction with the retaining nut to help hold the source ferrule affixed the central shaft assembly.

### BRIEF DESCRIPTION OF THE DRAWINGS

In the drawings, like reference numerals and letters refer to like parts throughout the various views, unless indicated otherwise.

FIG. 1 is a side cutaway sectional view of an exemplary hand piece and/or system according to an aspect of the present invention;

FIGS. 2A and 2B illustrates a side view of the exemplary hand piece and/or system of FIG. 1 with and without an exemplary retaining sleeve assembly;

FIG. 3 is a magnified view of an exemplary connection portion of the exemplary hand piece and/or system of FIG. 1;

FIG. 4 is a perspective view of another exemplary hand piece according to another aspect of the present invention;

FIGS. 5A and 5B are sectional disassembled views of portions of an exemplary hand piece according to an aspect of the present invention;

FIG. 6 is a perspective view showing an exemplary mechanism for attachment of a probe tip to a hand piece according to an aspect of the present invention;

FIGS. 7A-7C are perspective views of an exemplary hand piece with an exemplary alternative probe tip according to an aspect of the present invention,

FIG. 8 is a sectional cut away portion of the exemplary hand piece of FIGS. 7A-7C.

FIG. 9A and 9B respectively illustrate a disassembled hand piece and a close-up of a portion of that hand piece according to exemplary aspects of the present invention.

FIG. 10 illustrates an exemplary optical element according to an aspect of the present invention.

### DESCRIPTION OF THE PREFERRED EMBODIMENT

The present invention is predicated upon the provision of a hand piece for delivering light from a remote source/receiver instrument to tissue or biological matter such as an oral cavity or other body location for use in photodynamic therapy (PDT) such as photo-dynamic disinfection (PDD). It is also contemplated that the hand piece could be used for medical or other applications such as photo-activated anti-fungicidal therapy, photo-assisted tissue welding, photo activated melting or polymerization of therapeutic compounds, photo curing in light curing cement applications (erg., dental applications), medical laser applications (e.g., surgical cutting), medical ablation applications, photocoagulation in ophthalmology related applications, optical sensing applications, monitoring of optical processes or other applications.

The hand piece of the present invention can exhibit one or several desirable or advantageous characteristics, According to one aspect of the invention, the hand piece can be configured to survive repeated trips through an autoclave or chemical bath for serialization without substantial physical degradation or degradation of performance from optical components of the hand piece. For example, the hand piece can include an optical fiber that is protected and/or sealed (e.g., hermetically sealed) in a center shaft assembly for providing protection from heat, humidity or both to the optical fiber during serialization. For assisting in such protection or sealing, it can be desirable to use medical grade adhesives with high glass transition temperature for allowing the hand piece to endure repeat serialization in an autoclave or otherwise. One exemplary preferred adhesive is a two component epoxy adhesive sold under the tradename EPO-TEK 353 ND and commercially available from Epoxy Technology at 14 Fortune Drive in Billerica, Mass. 01821.

According to another aspect of the invention, the entire hand piece as a fully assembled unit (minus the source Fiber and the tip) can be put through an autoclave or a chemical bath for disinfection. It may occur that, after the autoclave, the surfaces on the proximal and distal ends (e.g., the surfaces of ferrules or the fiber running through the central shaft assembly) may need or require additional cleaning to restore ideal optical performance. Therefore, in one embodiment, both a proximal ferrule and a distal ferrule are integrated with the hand piece in a manner that allows one or both of the ferrules to be accessed for cleaning.

According to another aspect of the invention, the hand piece can include a body that protects the central shaft assembly in such a fashion that the central shaft assembly, the components on the proximal end of the hand piece or both are sufficiently isolated, during use of the hand piece or other, from any biological tissue (e.g., of a patient) that the assembly, the components or both do not need to be sterilized. It may be the case that the hand piece is designed such that only the tip and body need to be sterilized or disposed of, This means the optical components in the hand piece may not need to be designed to withstand serialization (e.g., in an autoclave), which can lengthen their service life and lowering their production cost.

According to another aspect, the hand piece can be designed to achieve low optical insertion/transmission losses. This can be accomplished, in part and/or in one embodiment, by matching characteristics of the fiber running through the central shaft assembly with the characteristics of the source fiber. As one example, tight axial tolerances between the two fibers can be maintained as well as maintaining control over the gap or distance between them. Holding these high tolerances while keeping cost low can be enhanced by the option of using industry standard fiber optics connectors. These designs also allow the ends of the fiber optics to be properly prepared (e.g. polished) in order to yield a long lasting low loss optical interconnect, as versus the variable loss and yield issues inherent with, for example, cleaved optical fibers.

As another example, stable optical performance over a range of thermal conditions can be achieved for the hand piece by matching the characteristics of the central shaft body to the ferrules and the fiber (e.g. by employing all glass and/or ceramic construction). By matching material characteristics, withdrawal of the optical fiber into the ferrule (e.g. piston), which can be caused by extreme temperature cycling of an autoclave, can be avoided or at least inhibited. In turn, reductions of component optical performance and/or lowering of the unit's lifetime can also be avoided or inhibited.

As yet another example, a mismatch of characteristics between the fiber and the central shaft and ferrules (i.e. brass shaft and steel ferrules with glass fiber) can be dealt with procedurally by soaking the assembly at autoclave temperatures, causing the fiber to permanently pull back into the ferrules slightly. This can be done during or after the cure process. The fiber ends can then be polished after temperature cycling, yielding a low loss assembly with enough “slack” at room temperature to accommodate future expansions caused by subsequent higher temperature events (i.e., trips so through an autoclave).

According to another aspect of the invention, the hand piece can have a modular design. In one embodiment, the hand piece disassembles into three pieces or sub-assemblies that are interchangeable between units. This allows a tray of them to be disassembled and run though the autoclave without the need for matching parts when re-assembling, It is also possible to combine a different body with the central shaft assembly to better fit needs of the application (e.g., the ergonomics of a particular task or the preferences of a technician). Moreover, it is contemplated that low cost, high performance, modularity or any combination thereof can be achieved by the option of utilizing standard, mass produced fiber ferrule components that are similar or identical to standard optical connectors.

According to another aspect of the present invention, the body section of the hand piece can be constructed with different contours to better fit the technicians hands or the type of treatment application. Moreover, in the case of a modular design, different body styles can be swapped on a single set of internal components. Thus, different body styles can be employed in conjunction with one optical fiber.

According to yet another aspect of the present invention, the design of the hand piece can allow it to be used with reusable tips or with tips that are single use (disposable). The features on either the distal end of the hand piece, the retaining sleeve or both can be designed to interface with retention features in either the tip, the body or both. These may include specific features that effect the retained component in such a way that removing the component “deactivates” the retention features, making the component significantly less useful for subsequent usages, thereby encouraging disposal and encouraging safe, single use behavior. Examples of other suitable tips, in addition to those discussed below, which may be used as replacements for the tips described herein or which may be used in conjunction with the present invention or features thereof are disclosed in U.S. patent application Ser. No. 11/397,768, filed Apr. 4, 2006, titled Optical Probe for Delivery of Light, which is expressly incorporated herein by reference for all purposes.

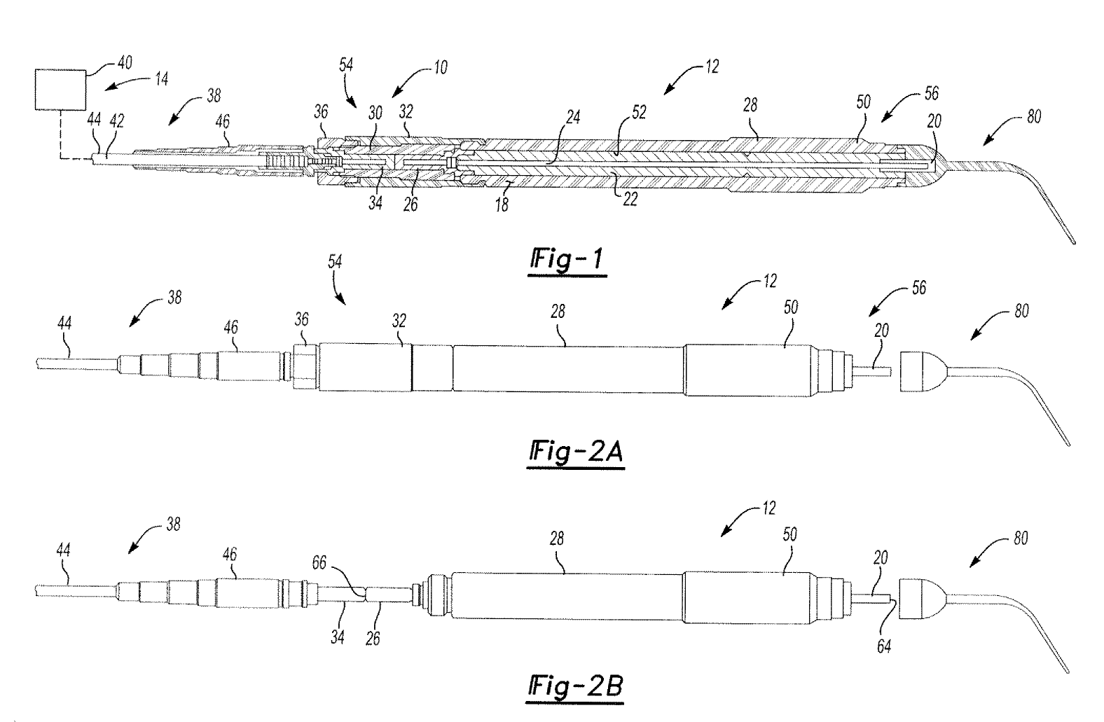

With reference to FIGS. 1, 2A and 26, an exemplary system 10 is illustrated, The system 10 includes a hand piece 12 and a light source assembly 14. The hand piece 12 includes a center shaft assembly 18 comprised of a one ore any combination of distal ferrule 20, a center shaft body 22, an optical fiber 24 and a proximal ferrule 26. The center shaft assembly 18 can be held in a body 28 of with a fastener (e.g., a set screw) that engages a groove in the center shaft body. An internal adapter 30 mates with the proximal ferrule 26 and is held in position by a retaining sleeve 32. A source ferrule 34 from the light source assembly 14 mates with the other side of the internal adapter 30 and is held in place with a retaining nut 36.

The source fiber assembly comprises the elements of the fiber optic cable 38 that bring light to and/or from the hand piece 12 and to and/or from a light source or instrument 40. The assembly can include, but is not limited to, a source fiber 42, a jacket 44, a strain relief 46, the ferrule 34 and the retaining nut 36. The retaining sleeve assembly comprises components that provide an interface for connecting the source fiber assembly to the body assembly. The retaining sleeve assembly can include, but is not limited to, the retaining sleeve 32 and the internal adapter 30. The body assembly comprises components that form a gripping section 50 of the hand piece 12, provide a conduit 52 through which the light can traverse back and/or forth between a proximal end 54 and a distal end 56 of the hand piece 12 and also provide an interface with the tip. Typically, the tip is an end effecter and can be configured for delivering light to the treatment area and/or for measuring certain characteristics about the treatment area.

Shown at the left in FIG. 1, the source fiber 42 is an optical fiber element that serves to conduct light from a source/receiver instrument 40 to the hand piece 12 and optionally from the hand piece 12 back to the source/receiver instrument 40. To serve this light conduit purpose, this fiber can be selected from multiple types of fiber optic, including, without limitation, silica (or glass), hard clad silica (HCS), polymer clad silica (PCS) and plastic fibers. Hollow core or liquid core waveguides may also be utilized. It is typical to protect the fiber by jacketing it in a protective sleeve. Moreover, it is within the scope of this invention that a multitude of different outer fiber jackets can be used, including, but not limited to, the wide variety of industry standard reinforced jackets. In oral PDD applications, this fiber is typically far enough removed from the patient that it does not need to be sterilized. However, it is worth noting that while most all silica and HCS will survive in an autoclave, other types of fiber tend to have very limited lives if they are ever exposed to such serialization techniques.

Although only a single fiber is shown in FIGS. 1-3, it is within the scope of this invention that either a single fiber with one core, a single fiber with multiple cores or a plurality of fibers may be used for the source optical fiber. The plurality of fibers may be a bundle of fibers acting as a single conductor or with individual fibers fulfilling separate purposes. As examples, without limiting the scope of this invention, some fibers may be used to provide light to the hand piece while others serve to conduct light back to the source/receiver instrument. Alternatively, or in combination with the preceding, various fibers may serve to conduct different wavelengths of light in either direction. Additionally, separately or in combination with the preceding the fiber bundle may include a coherent bundle of fibers that may, for example, be used for imaging purposes.

The source fiber or other fibers discussed herein may conduct radiation from any portion of the electromagnetic radiation spectrum. Of special interest are therapeutic wavelengths in the ultra violet, visible and near infra red portions of the spectrum. The source fiber or other fibers may emit one wavelength, a range of wavelengths of light or groups comprised of a combination of individual wavelengths and ranges of wavelengths. The source fiber or other fibers may conduct light to the hand piece and back to the source/receiver instrument. One group of wavelengths may be conducted outward from and another group of wavelengths back to the source/receiver instrument.

The fiber may be polished to a smooth surface that is either flat or has curvature, or the fiber may be cleaved to form a flat surface. The fiber may also have coatings on it to protect the fiber surface, lower reflection losses, or tailor the reflectivity for certain wavelengths. The fiber may also terminate in an optical element that serves to modify the way light is transmitted to the corresponding fiber in the hand piece, The fiber may have patterns etched in the surface to enhance transmission to form an optical element such, but not limited to, a diffractive optic or HOE. The fiber termination may also be a lens such as a ball lens or a graded index lens.

The source ferrule 34 provides a structure at the termination of the source fiber 42 and may also provide a location for the interface at the end of the fiber. The source ferrule 34 may be constructed from any of the broad number of industry standard fiber optic components, such as, without limitation, a stainless steel SMA ferrule as shown in FIG. 2. The source ferrule may have a standard shape or configuration or a custom shape or configuration (e.g., square or rectangular) and may have a non-symmetrical or a symmetrical shape (e.g., a cylindrical SMA ferrule), The source ferrule may also include features that serve to align the ferrule in a specific orientation (e.g., a keyed tab or a non-symmetrical and/or non-circular shape). The source ferrule may be constructed of any practical material, including but not limited to, glass, ceramic, metals and glass filled plastics depending upon desired dimensional tolerances, desired ability to hold the fiber secure inside of the ferrule better and desired durability for withstanding, for example, connect/disconnect cycles. Many commercially available ferrules are made from stainless steel or a zirconium based ceramic, however, the skilled artisan will recognize other materials that can be used depending upon the desired configuration,

The source ferrule can be configured to accommodate a single fiber or a plurality of fibers in a single ferrule. Without limitation, a bundle of plural (e.g., 5, 6, 7 or more) separate fibers can be packed into a ferrule with a single hole or an array of separate fibers placed linearly along a rectangular bar. The source ferrule may be comprised of a single ferrule or plurality of separate ferrules that may also be joined together in such a fashion as to comprise a single piece of material. Without limiting the scope of this invention, examples of a plurality of source ferrules may be a pair of SMA ferrules, one for outgoing light and one for returning light, Without limitation, an example of joined ferrules may be configure such that two or more cylindrical ferrules are joined together along a common line (i.e. glued or welded) or a component is fabricated from one piece with the appearance of a plurality of cylindrical ferrules joined together in a pattern such as linear array (i.e. a molded plastic piece).

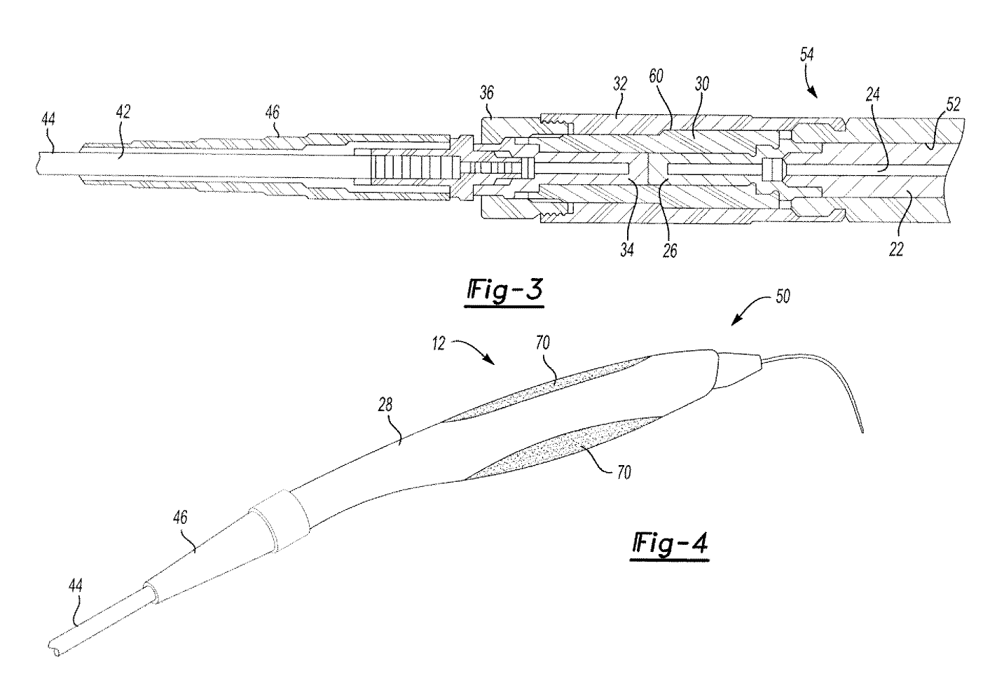

The source ferrule 34 in FIGS. 1, 2b and 3 is shown as an industry standard ferrule, specifically a SMA style ferrule. SMA ferrules are typically used in conjunction with a retaining nut and it may be convenient to consider it as part of a SMA Ferrule Assembly. As shown in detail in FIG. 1 and 3, when the SMA ferrule 34 is inserted into the retaining sleeve with corresponding external threads and/or within the adapter sleeve 30, the retaining nut 36 is used to engage the external threads and thereby functions to hold the SMA ferrule 34 securely in the adapter sleeve 32. Since it is within the scope of this invention that other fiber optics connectors can be utilized, it is within the scope of this invention that the function of the retaining nut may be preformed in a wide variety of other fashions besides threaded engagements. For example, without limitation, a bayonet style retaining barrel typical of an ST style may be utilized. Additionally, the function of the retaining nut could be accomplished by a combination of features on the source ferrule and/or the hand piece. An example of this, again without limitation, would be a configuration wherein the ferrule was inserted into the is hand piece and twisted to lock in place. Another non-limiting example would be wherein the source ferrule slides laterally into a pocket in the hand piece and is retained in a well aligned position by a spring loaded mechanism. Although retaining nuts are often made from aluminum or a steel, the skilled artisan will be able to select other desired materials. It is also within the scope of this invention that the source ferrule could be permanently affixed to the retaining sleeve assembly with an adhesive, although not necessarily desired.

With reference to FIG. 1, one embodiment of a retaining sleeve assembly is shown. The hand piece 12 showing the source fiber assembly ends in a source ferrule 34 that is located in close proximity to the proximal ferrule 26 on the body assembly. The internal adapter 30 aligns the two ferrules 26, 34, The retaining sleeve 32 is threaded or otherwise connected onto the body 28 and serves to clamp the internal adapter 30, the proximal ferrule 26 and central shaft assembly 18 to the body 28. The nut 36 threads or is positioned onto the internal adapter 30 and clamps the source fiber assembly to the hand piece 12.

In the embodiment of this invention shown in FIG. 3, the internal adapter 30 is a sleeve that serves to holds the fibers 24, 42 in the proximal ferrule 26 and the source ferrule 34 in close proximity and well aligned. On the source ferrule 34 side, the internal adapter 30 has external threads that engage with the retaining nut 36 to hold the source ferrule 34 firmly engaged. On the other end, the features in the internal adapter 30 accept or receive the s proximal ferrule 26. Also shown in FIG. 1 is the external engagement rim feature 60 on the internal adapter 30 that engages with a corresponding feature on the retaining sleeve 32.

The embodiment of the retaining sleeve 32 shown in FIG, 1 has internal threads that engage with external threads on the body 28. Also shown is the internal engagement rim feature that engages with the corresponding feature on the internal adapter 30. When the retaining sleeve 32 is threaded or otherwise located onto the body 28, the internal engagement rim 60 engages with the external engagement rim, thereby capturing the internal adapter 30. When the retaining sleeve 32 is tightened down to the body 28, this has the effect of securely retaining the internal adapter 30 onto the proximal ferrule 26. In this fashion, the embodiment shown in FIG. 1 shows how the source fiber assembly and the body assembly are held together in an aligned state by the combination of the internal adapter 30 and the retaining sleeve 32.

The embodiment in FIG. 3 shows the internal adapter and the retaining sleeve as separate components. It is also within the scope of this invention where the functions of the two components could also be fulfilled by a single component. In the embodiment shown in FIG. 1, this could be accomplished by adding a feature with internal threads to the distal end of the internal adapter that allows it to be threaded down to the external threads on the body.

The retaining sleeve may have features around a portion of its external surface to aid with both gripping it and removing it from the body. These features may include, without limitation, knurling, roughening, regions including a soft polymer material, protruding features (i.e. nubs) or prismatic features such a nut formed from a single or plurality of flat faces. The retaining sleeve may also possess ergonomic contours that enhance the comfort of the grip and aid with the establishing a secure grip during use. It is within the scope of this invention that the retaining sleeve may posses gripping and ergonomic features individually or in combination.

It is within the scope of this invention that the retaining sleeve may engage with the body in a different fashion than that shown in FIG. 3. For instance, this may include, without limitation, external threads on retaining sleeve engaging internal threads on the body, or the retaining sleeve engaging with the body with a bayonet style “twist to lock” mechanism. Other similar engagement mechanisms could also be employed. In a similar fashion, as discussed in the previous retaining nut description, the proximal end of the internal adapter may also engage with the source ferrule or the source fiber assembly. Note that in FIG. 3, the retaining sleeve assembly serves to hold the source ferrule assembly securely aligned with the proximal ferrule and holds the source fiber assembly securely to the body assembly. It is also within the scope of this invention that the central shaft assembly can also he held securely inside the body section by the clamping action between the retaining sleeve assembly and the body. As discussed in later sections, the central shaft assembly may also be held in the body by other means.

The retaining sleeve assembly can serve to convert from one style ferrule to another. An example of this, without limitation, would include mating first style source ferrule (e.g., an ST style source ferrule) with a second style source ferrule (e.g., an SMA style proximal connector). In such a case, there would be a first or SMA sized internal bore diameter for one part of the length of the retaining sleeve and a second or an ST sized bore for another part of its length, In addition, either the retaining sleeve assembly would need to have mechanisms (e.g., the posts and keyway) required to engage with the first second or ST style ferrule and a second mechanism (e.g., a bayonet interlock barrel) for engaging the second style ferrule. It is also within the scope of this invention that the retaining sleeve assembly could accept a single or a plurality of ferrules from either the source fiber assembly or the body assembly or both. The retaining sleeve assembly can also be configured accept arbitrary or different shaped or sized ferrules from the source fiber assembly or the body assembly or both, Examples of these type of ferrules include, but are not limited to, prismatic shaped ferrules, arrangements of joined ferrules, single ferrules with bundles of fibers or one set of ferrules for outward bound illumination and a second set of ferrules for return light.

It is within the scope of this invention that the components comprising the retaining sleeve assembly can be constructed of a wide range of possible materials. These include, but are not limited to, autoclave compatible materials (i.e., materials that can withstand autoclave conditions without significant degradation) such as stainless steel, brass, aluminum and other metal alloys as well as ceramics like Alumina or Zirkonia, or rigid polymers such as glass filled epoxy. It is also possible to form the components of the retaining sleeve assembly from materials (e.g., various plastics) that are not compatible with an autoclave. It is also within the scope of this invention that a mix a materials may be utilized. For example, without limitation, the internal adapter may be made of one material such as stainless steel and the retaining sleeve may be made of another material such as aluminum. It is also possible that an individual component may be comprised of more than one material. For example, again without limitation, the retaining sleeve may be constructed of aluminum with the gripping features formed from an inset of a compliant material such as silicon rubber.

The body assembly typically comprises the body component and one or any combination of the components that make up the central shaft assembly. As shown in the embodiment of FIG, 2B, the body assembly has a male ferrule 20, 26 extending outwardly or sticking out each end. Depending on the materials choices, the entire body assembly can be run through an autoclave while still connected with the retaining sleeve assembly or when disassembled, all without damaging any of sub-components. The unique configuration of components allows the optical surfaces 64, 66 on both ends of the device to be inspected and cleaned. This maintenance possibility allows the low loss optical performance of the device to be maintained even if foreign objects get deposited on the mating optical surfaces at 66. A benefit from the unique construction is that components from several hand pieces can be interchanged. This makes it simple to reassemble units if several components were sterilized at once and allows a single component to be upgraded or replaced. This would be an advantage if, without limitation, the optical surfaces 64, 66 of the central shaft assembly were damaged and needed replacement or if it was desired to switch one body style with another or if it is required to change the internal adapter in order to use one that is compatible with a different style of source fiber assembly. Note that it is possible to design the body and the retaining sleeve assembly so they are either constructed as a single piece or constructed so they can not be easily disassembled. This would allow effective serialization but could make it more difficult to inspect and clean the optical surfaces that were located down inside the shaft of the internal adapter.

The body 28 typically forms the outer shell of the hand piece 12 and can provide one or multiple functional attributes to the hand piece. For example, the body 28 can provide a gripping surface and shape. As another example, it protects the components inside of it, especially the optical components. As another example, it can provide a sterile barrier between the patient and the components of the hand piece. Yet another potential function of the body is to serve as a rigid base to hold all the various components rigidly together. Finally, another potential function is to provide a visually compelling form that focuses the attention of the patients and care providers on the brand and treatment technique being employed.

FIG. 4 shows an embodiment of the hand piece 12 where the body 28 section has been sculpted to provide a visually appealing form that provides a grip that is comfortable, low strain, secure or a combination thereof. The ergonomic contours can be designed to fit specific sized or shaped hands, allowing different users to assemble the hand piece with the body style that they find the most comfortable. The hand piece can also be designed with sections that have surface finish or surface features that aid in providing a secure grip. Without limitation, examples include roughened surfaces, ridges, patterns of nubs, patterns of divots, knurling, contoured finger intents, combinations thereof or the like, Sections of compliant material can also be included to aid with gripping. Without limitation, examples include sections of silicon rubber or even a silicon rubber sleeve encasing the entire body section. The compliant section can also have surface finish or surface features such as the aforementioned aid in providing a secure grip.

In FIG. 4, an embodiment of the hand piece 12 is shown where the body 28 is sculpted in an ergonomic fashion to provide a comfortable, low strain and secure grip. As can be seen, the body 28 is generally larger in diameter or bulbous toward the distal end of the hand piece 12 and this bulbous portion includes opposing compliant gripping surfaces 70 to aid in establishing a secure grip.

The design of the hand piece can, if desired, include a visual style that can be an important part of creating recognition for both the brand and the treatment by both the patient and the care provider. Such design features may include, without limitation, distinctive logos as shown in FIG. 4 and/or distinctive shapes, distinctive patterns of compliant inlays also shown in FIG. 4, and/or distinctive patterns of contrasting paint or other material, distinctive patterns of surface relief and even sections that light up when in use in a distinctive fashion. Creating body section that light up can be arranged by constructing portions of the body of translucent materials and arranging to have some of the outgoing or return light from the light source diverted into these sections. Distinctive patterns can be created by either the shape of the translucent sections or by overlaying opaque materials in distinctive patterns.

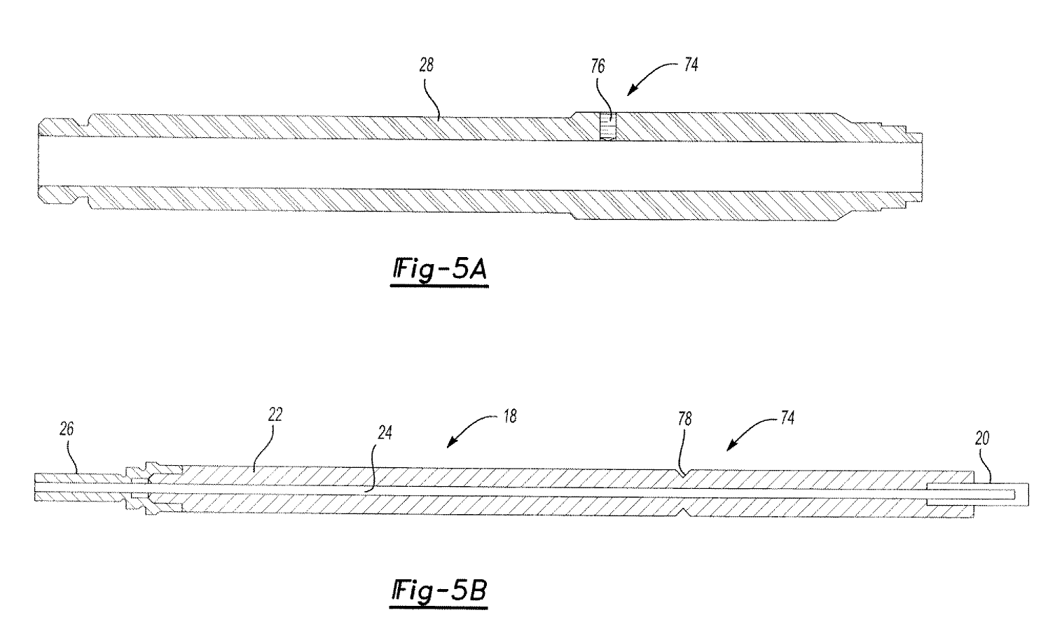

In the embodiment shown in FIGS. 1-3, the proximal side or end 54 of the body 28 interfaces with the components of the retaining sleeve assembly. The central shaft assembly can be contained and protected inside the body 28, with only the proximal ferrule 26 exposed on one side or end 54 and the distal ferrule 20 on the other. The act of engaging the body to the retaining sleeve assembly can serve to hold all the parts clamped securely together. Alternately or in combination with the aforementioned, the central shaft assembly 18 can be held into the body 28 by a retention mechanism 74 such as the set screw 76 shown in FIG. 5 The set screw 76 is held by threads in the body 28 and it's tip engages the central shaft assembly. If desired, the set screw can engage a retention feature 78 such as the groove shown on the central shaft assembly in FIG. 5. Note that the potential variations in the specific design for how the body 28 engages with the retaining sleeve assembly have been discussed earlier.

The body 28 can be constructed of a wide range of potential materials, If the body will be sterilized in an autoclave, then materials compatible with high heat and humidity should be chosen. Without limitation, examples are metals like stainless steel and aluminum, or ceramics like Alumina or Zirkonia or durable polymers such as glass filled epoxy or some silicon rubber compounds. If the body is to be chemically sterilized, then materials with low reactivity should be chosen. Without limitation, examples are plastics like polycarbonate, polymers such as silicon rubber compounds or metals such as stainless steel. The body can also be formed of ceramic compounds to survive both autoclave and chemical serialization. The body can also be formed of combinations of multiple materials, such as, without limitation, silicon rubber gripping inserts in a stainless steel structure, aluminum structure with an ergonomic silicon rubber over-molded sleeve, or even a ceramic structure with a threaded aluminum insert in the proximal end to engage with the retaining sleeve assembly. If the body section is to be disposable, then the body should be made of low cost materials such as plastics.

As shown in FIGS. 1 and 5, the central shaft assembly 18, the body 28 or both can substantially encase or contain the optical component 24 that runs down the length of the hand piece 12 as well as the components that interface with the source ferrule and the tip. Components that protect the optics during assembly and form a seal (e.g., a hermetic seal) around the optics can also be included. FIG. 5 shows an embodiment where the central shaft body is combined with the proximal and distal ferrules 20, 26 to form the central shaft assembly, a rigid, sealed (e.g., hermetically sealed) unit that protects the optical fiber 24. If a hermetic seal is desired, the fiber can be for example, soldered in a metal sleeve. As shown in FIGS. 1 and 5, the central shaft assembly 18 is inserted into the proximal end 54 of the body 28. It is held in place either by the clamping action of the retaining sleeve 32 against a feature on the base of the proximal ferrule 26 or by the set screw 76 engaging in the retention groove 78, or both. One significant advantage of this configuration is both ends of the central shaft assembly 18 are male fiber ferrules 20, 26 that are easy to manufacture precision ends as well as to inspect and clean.

Note that in FIGS. 1-5 the distal ferrule 20 is shown as bare ferrule inserted into the central shaft body, while the proximal ferrule 26 has a base section body that engages over the end of the central shaft body 22, It is within the scope of this invention that either style of ferrule can be used on either end, although this may effect which end of the body the central shaft assembly can be inserted into. It is also within the scope of this invention that the end of the central shaft assembly may engage on a lip provided at the distal end of the body. There may also be a seal provided between the body and the central shaft assembly at either or both ends in order to reduce the opportunity for contaminating material to work in between the two, Such a seal can be provided through the use of medical grade adhesives as discussed herein or otherwise.

The proximal ferrule is typically configured to hold a single or plurality of optical elements (e.g., fiber[s]) aligned with corresponding optical element(s) (e.g., fiber[s]) in the source fiber assembly. The previous discussion about ferrule shapes, materials and number of optical conductors in the source fiber assembly also applies to the proximal ferrule, For example, a bare barrel ferrule and one with a base section could be chosen depending upon the desired configuration for the overall hand piece. It should be noted that for high power applications (e.g., delivery of laser power in excess of 1 watt), it may be more appropriate to utilize metal ferrules due to their ability to better withstand higher temperatures compared to ceramic or polymer ferrules.

The distal ferrule is intended to interface or receive the single or plurality of optical elements (e.g., fiber[s]) running down the central shaft body with the optical section of the tip. The previous discussion about ferrule shapes, materials and number of optical conductors in the source ferrule assembly also applies to the distal ferrule. For example, a bare barrel ferrule and one with a base section could be chosen depending upon the desired configuration for the overall hand piece. Again, it should be noted that for high power applications (e.g., delivery of laser power in excess of 1 watt), it may be more appropriate to utilize metal ferrules due to their ability to better withstand higher temperatures compared to ceramic or polymer ferrules,

The embodiment shown in FIG. 5 has a single optical fiber running between the proximal and distal ferrules, although multiple fibers or light conducting elements may be employed. It is within the scope of this invention that a wide variety of different light conducting elements could be utilized. Although the optical fiber is often referenced herein, it should be understood that such fiber may be replaced by any of the light conducting elements discussed herein or other art disclosed elements. Without limitation, examples are glass clad silica fibers, hard clad silica fibers, polymer clad silica fibers and polymer fibers. The optical fiber may have cylindrical shapes or be composed of arbitrary or alternative cross sections (e.g., square, triangular or other extrusion shapes). The optical fiber may have a cladding on it or may be clad only in the media inside the central shaft body. Note that the fibers composed of glass and/or silica glass tend to be rugged and resistant to autoclave type or chemical serialization, whereas many of the polymer fibers are not as resistant to high temperatures, high humidity or harsh chemicals.

The optical fiber may conduct radiation from any portion of the electromagnetic radiation spectrum. Of especial interest are therapeutic wavelengths in the ultra violet, visible and near infra red portion of the spectrum. The optical fiber may transmit one wavelength, a range of wavelengths of light or groups comprised of a combination of individual wavelengths and ranges of wavelengths. The optical fiber may conduct light to the tip and back to the source fiber. One group of wavelengths may be conducted outward and another group of wavelengths back,

The ends of the fiber may be treated the same or have different characteristics. The fibers may be polished to a smooth surface that is either flat or has curvature, or the fiber may be cleaved to form a flat surface. The fiber may also have coatings on it to protect the fiber surface, lower reflection losses, or tailor the reflectivity for certain wavelengths. The fiber may also terminate in an optical element that serves to modify the way light is transmitted from the fiber. The fiber may have patterns etched in the surface to enhance transmission to form an optical element such, but not limited to, a diffractive optic or HOE, The fiber termination may also be a lens such as a ball lens or a graded index lens.

Much the same as mentioned with the source or input fiber, the optical fiber may comprise either a single fiber element, fiber with multiple cores or a plurality of fibers may be used. The plurality of fibers may be a bundle of fibers acting as a single conductor or with individual fibers fulfilling separate purposes. Alternatively, or in combination with the preceding, various fibers may serve to conduct different wavelengths of light in either direction. Additionally, separately in combination with the preceding, the fiber bundle may include a coherent bundle of fibers that may, for example, be used for imaging purposes. A coherent bundle of fibers is a bundle of fiber elements that is capable of reproducing an image on its distal end that corresponds to an image that is focused on its proximal end.

In addition to singular or multiple optical conductors passing straight through the central shaft assembly, it is also within the scope of this invention that there may be other optical elements inside the center shaft body that serve to redirect or combine the light into new configurations. Without limitation, an illustrative example is the inclusion of a mechanical or fused “Y” coupler used so there is a single fiber on the proximal end and a pair of fibers on the distal end. In such an embodiment, the pair of fibers would share the light that was transmitted through single fiber and the single fiber would carry a combination of the light transmitted through the pair of fibers. This concept may also include almost any number (e.g. 2, 3, 4, 5, or more) of fibers on the proximal side and end up with almost any number (e.g. 2, 3, 4, 5, or more) of fibers on the distal end that may be the same as the number on the proximal end or may be a different number of fibers (e.g. a reduction or an increase in the fiber count). If a pair of fibers was used on either end, this would make a fused or mechanical “X” fiber splitter, sometime referred to as a “coupler”.

Straight through and coupled fibers may also be used in combination. Without limitation, an example is a pair of fibers on the proximal end, where a first fiber is configured to deliver therapeutic light to the treatment area and a second fiber is configured to return sensing light to the source/receiver instrument. The first fiber could be carried straight through to the distal end where it delivers its light into the Tip. The second fiber could be coupled to an array of multiple (e.g., six) fibers that surround the first fiber at the distal end. In this fashion, the arrangement of multiple fibers could be used to collect diffuse return light from the tip and ensure that a portion of that light made it into the second fiber that returns light to the source/receiver instrument for measurement and sensing purposes.

The fiber couplers may have directional spectral characteristics where wavelengths of light get split so that some wavelengths travel into one or more fibers and the rest travel into a different one or more fibers. Without limitation, an example is a 2:1 coupler where there are two fibers on the proximal end and a single fiber on the distal end. The therapeutic wavelength(s) may be introduced into a first proximal fiber where they are transmitted through the coupler into the single fiber and to the tip. The return light from the Tip may be routed so any light not in the band of therapeutic wavelengths are routed into the second proximal fiber. There are several mature techniques used for such wavelength splitting with fibers that include the used of filters, gratings or specific fusing geometries.

The embodiment in FIG. 5 depicts the central shaft body 22 as a cylindrical shape, which is easy to manufacture. However, it is within the scope of this invention that the central shaft body can have any arbitrary or predetermined cross sectional shape, including but not limited to oval, rectangular or even a pair or more of axially adjoined cylinders.

If a design goal of the hand piece is to make it able to survive serialization via chemical or autoclave techniques, then it is useful to make the central shaft assembly into an assembly (e.g., a hermetic assembly) that protects the optical fiber, only exposing the distal and proximal end surfaces of the fiber. This keeps the integrity of the optical fiber from degrading and maintains the low loss transmission characteristics of the hand piece. However, during thermal cycling in autoclave, the materials in the central shaft assembly can undergo significant thermal expansion. If there is thermal expansion mismatch between the optical fiber and central shaft body, then undesirable tension can be exerted on the optical fiber, potentially degrading or destroying it. As an illustrative example, consider an optical fiber 85 mm long. If the central shaft body is constructed from aluminum, then there can exist a 15 ppm/° C. (parts per million per degree Celsius) thermal expansion mismatch. For autoclave temperatures of 250° C., the central shaft body has expanded 0.25 mm more than the optical fiber. This may have the effect of retracting the optical fiber into or out of one or both of the ferrules, creating a large gap that may increase the optical transmission loss of the hand piece. Or, it may simply break the fiber if it can not stretch enough.

As such, it is within the scope of at least one embodiment of this invention that a newly invented technique for dealing with the thermal mismatch may be employed either in the design or the manufacturing process. To solve the thermal expansion issue in the manufacturing process, the central shaft assembly is constructed, but the fiber is left protruding out of each end of the ferrules a short distance. An un-cured adhesive is used to seal the optical fiber into the ferrules, then the assembly is elevated to the autoclave temperature for a long enough duration so that the adhesive can set or cure while the materials are in their expanded state. Once cooled down, there will be a small amount of “slack” optical fiber inside the central shaft assembly that will act as a buffer against future thermal expansion. In this state, end treatments (e.g. polished the fiber ends) can now be applied to the optical fiber that will be less subject to pull back and damage during temperature cycling.

Another new manufacturing technique has been proven to solve or alleviate thermal mismatch issues in a similar fashion to the elevated cure technique. In the case where a cured adhesive or even a glass solder joint exists between the optical fiber and the central shaft body, it has been shown that with repeated, short thermal cycling, the fiber gradually but permanently pulls back into the ferrules, without damaging the seals, which may or may not be hermetic seals. An example of how this technique may be utilized starts with an optical fiber that is glued or adhered into the central shaft assembly, but the ends are left long so they protrude a distance from the ends of the ferrules. The fiber needs to extend at least as much as the expected thermal mismatch (e.g. more than 0.5 mm), however it is practical that the length be 10-20 mm to facilitate further handling steps. The Assembly can then be repeatedly cycled between room temperature and autoclave temperature (e.g. 20 cycles of 15 minutes each full cycle), After repeated cycling, the fiber has either stretched or retracted, or both, to create the same “slack” condition referred to with the elevated cure. The ends of the fiber can now be prepared, e.g. by cleaving or polishing level with the end of the ferrule.

Metal components for the ferrules and central shaft body can be used to produce strong assemblies, which may or may not be hermetic, but they tend to have thermal expansion coefficients greater than fiber optic elements. However, another option exists to alleviate the thermal cycling issues. If the in materials in the central shaft assembly are chosen that have closely matched thermal expansion properties, then the effect of temperature cycling can be reduced or negated. It is most important that the fiber optic and the central shaft body match thermal properties closely, but some additional gain can be gained from matching the thermal properties of the ferrules to the fiber as as well. Without limitation, examples of the matching materials are the use of glass, ceramics, composites (i.e. fiber glass), glass filled epoxies or mixtures of the like. For example, without limitation, ceramic ferrules are fairly common and they could be matched with a ceramic or glass central shaft body.

In a preferred embodiment, at elevated temperature, the central shaft body expands or extends along its length a first distance and the optical fiber expands or extends along its own length a second distance and the first distance is within 1 mm, more typically within 0.5 mm and even more typically within 0.1 mm of the second distance. The elevated temperature is a temperature typical of an autoclave (e.g., between about 100° C. and about 300° C. or between about 200° C. and about 300° C. ).

As mentioned previously, the optical fiber can be glued into the ferrules. Without limitation, examples of appropriate adhesives are epoxies and urethanes. It is also possible to use glass solder compounds to seal the optical fiber into the ferrules, It is also possible to use metal solders to seal the optical fiber into the ferrules, but it may be desirable to create a metal “seed” layer on the non-metallic components (i.e. the fiber optics) in order to promote adhesion, The glass and metal solder compounds can be used to create seals by application of various forms of heat, including but not limited to laser energy, infrared radiation or exposure to an oven. One practical consideration is that the glass or metal solder compounds, if necessary, should remain mechanically stable at autoclave temperatures.

The ferrules and the central shaft body can also be sealed together using adhesives, including, without limitation, epoxies, urethanes and elastomer sealant (RTV) compounds. Glass and metal solder compounds can also be utilized, with similar requirements for the processing steps. In the case of metal ferrules and a metal central shaft body, it is also possible to create a direct weld using high quality welding techniques such as, but not limited to, laser welding, MIG welding and TIG welding. A swaged connection could also serve to securely join ferrules to the central shaft body if both are made of metal or of polymer materials or of combinations of the like. One typical method of forming a swaged connection is to crimp the outer of two concentric tubes so the outer tube collapses down to form a mechanical connection with the inner tube. In a similar fashion, press fit connections can also be used to form secure connections between ferrules and the central shaft body. In swaged and press fit connections, a sealing agent, exemplified by an application of an adhesive such as an epoxy, a urethane or a RTV compound, may be employed to assure tighter seals an assist in forming a seal (e.g., a hermetic seal).

It is also possible that a “one piece” central shaft assembly can be produced by molding the geometry of the ferrules and the central shaft body directly onto the optical fiber. This can be accomplished using several materials, including, but not limited to, ceramics, composites and glass filled epoxies. In such a case, the materials would be formed around fiber and cured. Then the single piece units could be processed to create the precision fiber ends and any other critical geometric features required. It is practical to make the entire hand piece or at least the body assembly disposable if the production cost of the single piece design can be made low enough.

The embodiment shown in FIG. 5 has a set screw 76 that threads into the body 28 and engages in the retention groove 78 to securely capture the central shaft assembly 18 in the body 28. FIG, 5 shows the retention groove 78 as a radial groove with a triangular cross section and the set screw 76 having a pointed end. It is contemplated that there can be a slight axial offset between the axis of the set screw hole and the bottom of the groove 78. The result is that when the set screw 76 is advanced forward into the hole, the s distal side of the slanted tip engages with the slanted wall on the distal side of the retention groove 78, forcing the central shaft assembly 18 to slide towards the distal end of the body 28. This has the effect of firmly engaging the body 28 on the proximal ferrule 26 against the body 28, creating a secure and rigidly coupled body assembly.

It is also within the scope of this invention that the set screw may have other styles of tips, including, without limitation, a radius tip, a polymer tip, a spring loaded ball tip, a soft metal pad on the tip. The shape of the retaining groove may also have other profiles, including, without limitation, radius profiles or square profiles. Further, the retention groove does not have to extend radially around the circumference of the central shaft body, it may instead be a hole or a divot that engages with the set screw. Such a feature would provide a singular alignment state would serve to rotationally align the central shaft assembly inside the body, which could be an advantage if there was a specific rotation keying desired anywhere in the hand piece. A non-limiting example of where this keying would be useful is if there were two optical fibers in the central shaft assembly, one for therapeutic light and one for return light. The keying feature could ensure that these fibers were lined up with the corresponding fibers in the source fiber assembly or with features in the tip. The set screw is an optional design element unless other otherwise stated. The retaining sleeve assembly also can securely clamp the central shaft assembly into the body, It is also possible to put internal threads on the body and external threads in the central shaft assembly so that the two are securely engaged when threaded together.

It is also possible that the functions of the body and the central shaft body can be combined into a single component. Examination of FIG. 1 indicates that the proximal and distal ferrules 20, 26 could be mounted directly in the body without substantially changing any of the other aspects of the hand piece 12. This combined part can be less expensive to construct. The two components could also be constructed separately and permanently joined with the application of an adhesive. As with the single piece design, if the production cost of the degenerate case can be made low enough, then the entire body assembly or even the entire hand piece could be made as a disposable unit. If this were to occur, there would be no need for autoclave serialization and cheaper materials could be utilized.

FIG, 1 shows an embodiment where a tip 80 is held onto the body assembly by friction and vacuum pressure, The act of pressing the tip xx onto the body assembly will displace air from the between the mating surfaces of the tip 80 and the body assembly. If the tolerances between the tip and the body assembly are tight enough, air can not easily slip back into the pocket, so the tip is securely retained by air pressure.

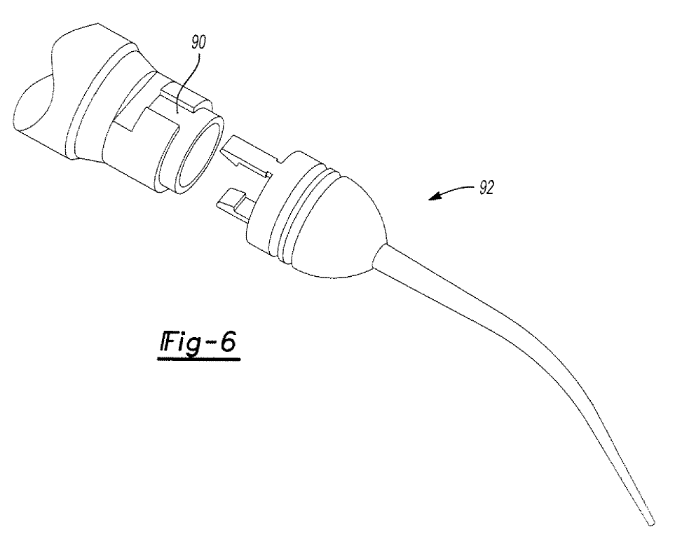

Tip retention can also include mechanical interlocking features in the Body Assembly that engage with corresponding features in the tip. In the embodiment shown in FIG, 6, the body assembly has an axial slot 90 that accepts the arms extending off the proximal end of the tip 92. When the tip has been pressed on far enough, the teeth on the end of the arms snap down and engage in the slot that is perpendicular to the axial slot. In this fashion, the features in the body section interlock with features on the tip to prove mechanically secure assembly. It is within the scope of this invention that the interlocking features in the body section can be different that those shown in FIG. 6. Without limitation, examples of other interlock features include threads, other slot geometries, posts, holes, and arms similar to the ones shown on the tip in FIG. 6. In addition, it is possible to form a collet features in the body assembly so that when the retaining sleeve is tightened down, it has the results of tightening the collet and establishing clamping grip on the tip.

Another embodiment or aspect of the hand piece is shown in FIG, 8. In this embodiment, the source ferrule, the proximal ferrules and the internal adapter are omitted. A source fiber 100 is connected directly to the distal ferrule 102 on the central shaft assembly 103. A strain relief boot 104 engages onto a stop feature 106, which is, in turn, inserted into the end of the central shaft body. A retaining sleeve 108 is captured onto the central shaft assembly due to an internal lip 112 that can be caught between the larger diameter of the central shaft body and the larger diameter of the stop feature 106. Instead of the threaded engagement shown in FIG. 3, the body 116 in this embodiment is shown connecting to the retaining sleeve 108 using interlock features similar to those shown for capturing the tip in FIG. 6. A spring 120 serves to push the retaining sleeve towards the proximal end of the central shaft assembly, effectively pulling the body with it. When the combined body tip is engaged with the retaining sleeve, the spring has the effect of pulling the tip down onto the distal ferrule 102.

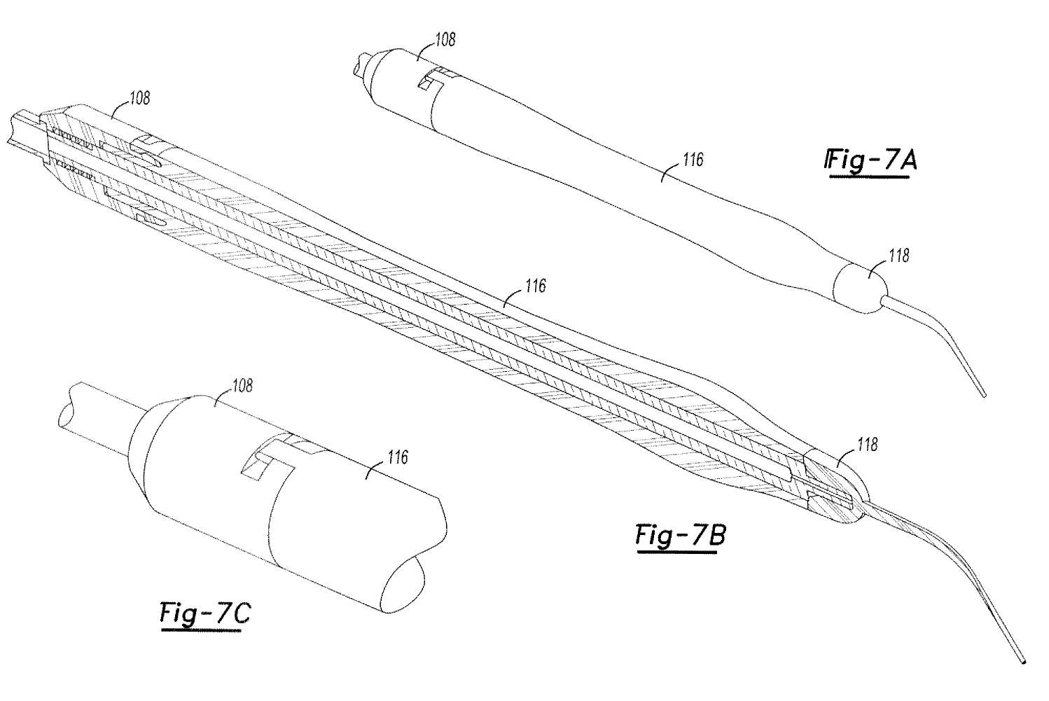

As shown in FIGS. 7A-7C, there is an embodiment of the hand piece where the body 116 and the tip 118 have been combined into a single, disposable element that completely covers and protects the majority of the hand piece. As shown in FIG. 7B, the combined body 116 and tip 118 slide over the central shaft assembly and engage with a modified retaining sleeve. As shown in FIG. 7C, a retaining sleeve 108 has interlock features similar to those at the distal end of the body in FIG. 6.

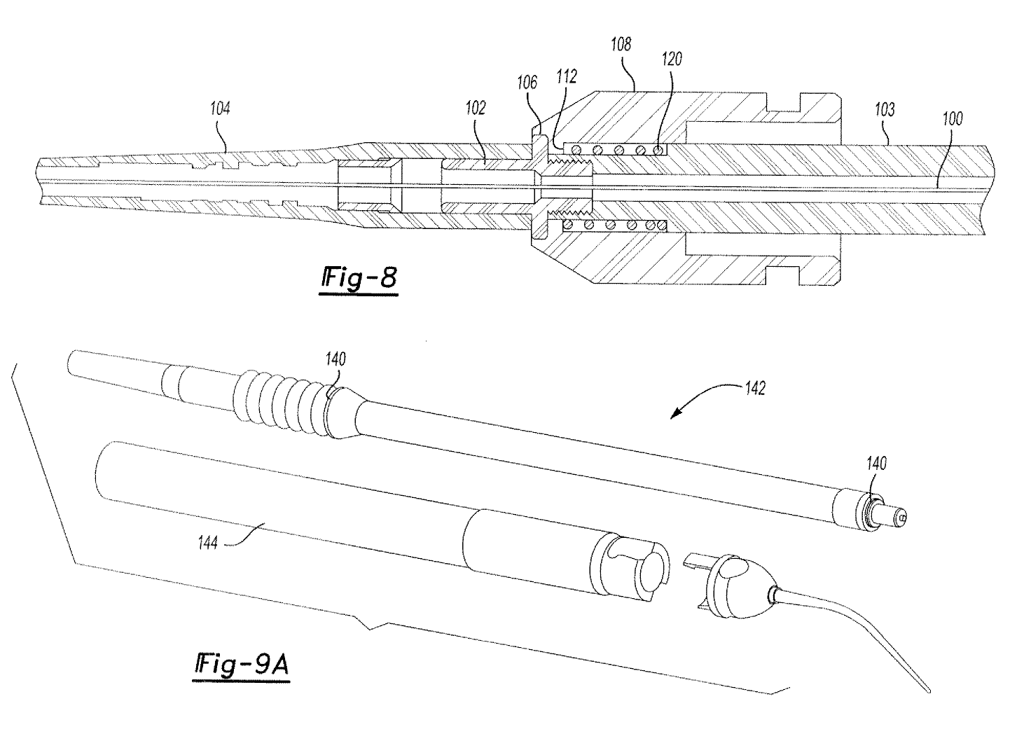

FIG. 8 shows a close up of the retaining sleeve 108 depicted in FIGS. 7A-7C showing how it is captured between the central shaft assembly and the stop feature. When the retaining sleeve 108 is engaged with the body 116, the spring 120 forces the body 116 onto the central shaft assembly until the body 116 tip 118 combination bottoms out or the retaining sleeve 108 hits the stop feature.

FIG. 8 shows the stop feature 106 press fit into the proximal end of the central shaft body. It is also within the scope of this invention that the stop feature may also be connected by other methods, including, without limitation, threaded connections, glued connection, soldered connection, or welded connection. It may also be pressed onto the outside of the reduced diameter section at the proximal end of the central shaft body.

It is within the scope of this invention that the tip can also be connected to the body also using interlock features similar to those shown in FIG. 6. This would allow the body to remain on the hand piece while tip was replaced or allow both of them to be changed, Advantageously, the body and the tip may be molded together as a single disposable piece. The combined body/tip covers the entire central shaft assembly and engages with the retaining sleeve, Since the entire central shaft assembly is protected from contamination, it does not need to be sterilized. Therefore, it does not necessarily have to be designed to withstand the harsh environment of an autoclave. This can simplify the design, allow for less expensive materials, lower the production cost and reduce the labor burden for the care giver. Additionally, it can decrease the chance of excess losses occurring due to contamination getting bake onto the end of the distal ferrule.

A combined body and tip would ideally be molded in one step from the same material. However, as previously mentioned, it is within the scope of this invention that they are originally formed as two separate parts that are physically combined. Methods of combination are, without limitation, press fitting, engaging physical interlock features, gluing together, melting together or ultrasonic bonding. It is also within the scope of this invention that the body and the tip can be formed from two separate materials. For example, without limitation, the body and tip can be formed of polycarbonate but the ergonomic gripping region can formed as a over-molding of silicon rubber. The introduction of disposable body sections allows the introduction of a range of different ergonomic styles in the same product line, allowing the care giver to easily choose the style that fits their hand and application.

There are adaptations, combinations modifications to the invention that are not specifically mentioned but would, in the light of this disclosure, now be apparent to one skilled in the art of mechanical design and are therefore clearly within the scope of this invention. An example would be to utilize the physical interlock features shown in FIG. 7C as the retention mechanism for the embodiment shown in FIG. 3. Another would be to combine the features of the embodiment in FIG. 7A-7C with that of FIG. 1 in a fashion that resulted in a disposable combined Body/Tip mounted on a hand piece where the central shaft assembly could still be removed from the source fiber assembly and run through an autoclave for serialization.

Additional or alternative features that may be used in the practice of this invention are also shown in FIGS. 9A and 9B. As can be seen, a hand piece is shown to include a seal 140 (e.g., an elastic O-ring) at a proximal end and distal end of a central shaft assembly 142. Advantageously such seals 140 can seal between an outer body 144 and the central shaft assembly 142.

It is contemplated that for the embodiment of FIGS. 9A and 98 as well as the other embodiments, that sealing between the central shaft assembly and the outer body can be sufficient such that it becomes unnecessary to autoclave or s otherwise sterilize the central shaft assembly and only the outer body need be autoclaved or otherwise sterilized.

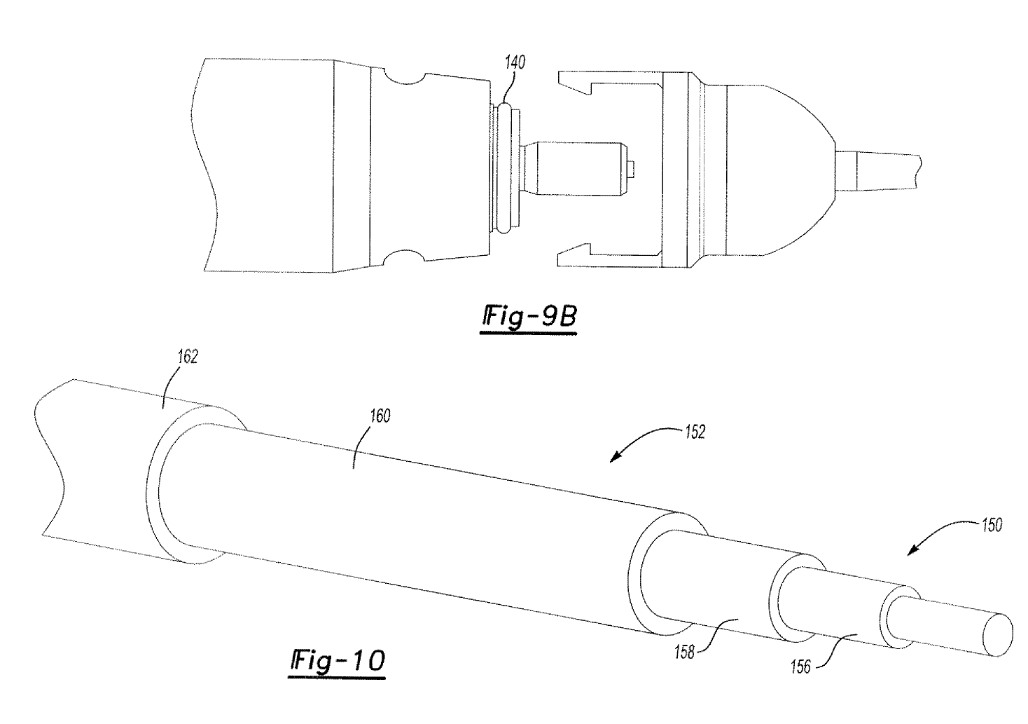

With reference to FIG. 10, it is contemplated for the embodiment of FIGS. 9A or 9B that metallized fibers may be employed in the practice of the present invention. FIG. 10 shows a fiber 150 having a metal coating 152 (e.g., a film). The particular fiber 150 shown includes a coating 152 with multiple layers, each layer of a different metal or other material (e.g., a titanium layer 156, a nickel layer 158, a gold layer 160 and a buffer coat layer 162). It is additionally contemplated, however, that a single layer may also be used and the single layer or any of the layers could be mixtures of metal and/or other materials. Such a coating can have a thickness between about 100 and about 2000 nm although it may be thicker or thinner.

Unless stated otherwise, dimensions and geometries of the various structures depicted herein are not intended to be restrictive of the invention, and other dimensions or geometries are possible. Plural structural components can be provided by a single integrated structure. Alternatively, a single integrated structure might be divided into separate plural components, In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention,

The preferred embodiment of the present invention has been disclosed. A person of ordinary skill in the art would realize however, that certain modifications would come within the teachings of this invention. Therefore, the following claims should be studied to determine the true scope and content of the invention.

What is claimed is:

1. A hand piece suitable for the delivery of light in medical applications, the hand piece comprising:
a central shaft assembly having a proximate end and a distal end, the shaft assembly including:
i. an proximal ferrule;
ii. a distal ferrule; and
iii. an optical element extending between the proximal and the distal ferrule;
an outer grippable body for substantially surrounding the central shaft assembly.

2. A hand piece as in claim 1 further comprising a retaining sleeve at the proximate end of the shaft assembly.

3. A hand piece as in claim 1 wherein the optical fiber is hermetically sealed within the central shaft assembly, the body or both such that the hand piece can be sterilized without substantial degradation to the optical fiber.

4. A hand piece as in claim 1 wherein the central shaft assembly further includes a center shaft body.

5. A hand piece as in claim 1 further comprising:
a tip wherein at least the outer body and the tip are disposable and are attachable and removable from the distal ferrule and the optical fiber and wherein the body and the tip cover the distal ferrule and the optical fiber when attached.

6. A hand piece as in claim 1 wherein one or more characteristics of the optical fiber are configured to match one or more characteristics of a source fiber that delivers light to the optical fiber.

7. A hand piece as in claim 6 the one or more characteristics of the optical fiber and the source fibers include diameter of the optical fiber and source fiber and tolerance of the diameter of the optical fiber and source fiber.

8. A hand piece as in claim 1 wherein the proximate ferrule, the distal ferrule or both extend outwardly beyond the outer body of the hand piece thereby allowing for access to the proximate ferrule, the distal ferrule or both, which, in turn, allows for cleaning, polishing or both thereof.

9. A hand piece as in claim 1 wherein the body, the ferrules, the optical fiber or any combination thereof are configured such that exposure of the hand piece to elevated temperature minimally affects the optical performance of the hand piece.

10. A hand piece as in claim 9 wherein the temperature expansion/contraction characteristics of the material of the body are substantially matched to the temperature expansion/contraction characteristics of the optical fiber, the ferrules or both.

11. A hand piece as in claim 4 wherein the hand piece is soaked at an elevated temperature such that the optical fiber is located in the central shaft body, the outer body or both in an manner that provide enough slack to the optical fiber to allow future exposure of the hand piece to elevated temperature without significantly stretching the optical fiber.

12. A hand piece as in claim 1 wherein the proximate ferrule is provided a standard size that allows connection to a source fiber have a standard size connector.

13. A hand piece suitable for the delivery of light in medical applications, the hand piece comprising:
a central shaft assembly having a proximate end and a distal end, the shaft assembly including:
i. a proximate ferrule;
ii. a distal ferrule;
iii. an optical fiber extending between the proximal ferrule and the distal ferrule; and
iv. a central shaft body substantially surrounding the optical fiber;
an outer grippable body for substantially surrounding the central shaft assembly; and
a retaining sleeve for assisting in connecting and disconnecting the hand piece to a source optical element;
wherein, for protecting the optical fiber from degradation during serialization, either i) the optical fiber is hermetically seal within the hand piece, ii) the optical fiber is provided with slack or iii) temperature expansion/contraction characteristics of the material of the outer body, the central shaft body or both are matched to the temperature expansion/contraction characteristics of the optical fiber, the ferrules or both within the hand piece.

14. A hand piece as in claim 13 further comprising:
a tip wherein at least the outer body and the tip are disposable and are attachable and removable from the distal ferrule and the optical fiber and wherein the body and the tip cover the distal ferrule and the optical fiber when attached.

15. A hand piece as in claim 13 wherein one or more characteristics of the optical fiber are configured to match one or more characteristics of a source fiber that delivers light to the optical fiber and wherein the one or more characteristics of the optical fiber and the source fibers include diameter of the optical fiber and source fiber and tolerance of the diameter of the optical fiber and source fiber.

16. A hand piece as in claim 13 wherein the proximate ferrule, the distal ferrule or both extend outwardly beyond the outer body of the hand piece thereby allowing for access to the proximate ferrule, the distal ferrule or both, which, in turn, allows for cleaning, polishing or both thereof.

17. A hand piece as in claim 13 wherein the hand piece is soaked at an elevated temperature such that the optical fiber is located in the central shaft body, the outer body or both in an manner that provide enough slack to the optical fiber to allow future exposure of the hand piece to elevated temperature without significantly stretching the optical fiber.

18. A hand piece as in claim 13 wherein the proximate ferrule is provided a standard size that allows connection to a source fiber have a standard size connector.

19. A hand piece suitable for the delivery of light in medical applications, the hand piece comprising:
a central shaft assembly having a proximate end and a distal end, the shaft assembly including:
i. a proximate ferrule;
ii. a distal ferrule;
iii, an optical fiber extending between the proximal ferrule and the distal ferrule; and
iv, a central shaft body substantially surrounding the optical fiber;
an outer grippable body for substantially surrounding the central shaft assembly; and
a retaining sleeve for assisting in connecting and disconnecting the hand piece to a source optical element;
wherein, for protecting the optical fiber from degradation during sterilization, either i) the optical fiber is hermetically seal within the hand piece, ii) the optical fiber is provided with slack or iii) temperature expansion/contraction characteristics of the material of the outer body, the central shaft body or both are matched to the temperature expansion/contraction characteristics of the optical fiber, the ferrules or both within the hand piece;
wherein one or more characteristics of the optical fiber are configured to match one or more characteristics of a source fiber that delivers light to the optical fiber and the one or more characteristics of the optical fiber and the source fibers include diameter of the optical fiber and source fiber and tolerance of the diameter of the optical fiber and source fiber;
wherein the proximate ferrule, the distal ferrule or both extend outwardly beyond the body of the hand piece thereby allowing for access to the proximate ferrule, the distal ferrule or both, which, in turn, allows for cleaning, polishing or both thereof; and
wherein the proximate ferrule is provided a standard size that allows connection to a source fiber having a standard size connector.

20. A hand piece as in claim 19 further comprising:
a tip wherein at least the outer body and the tip are disposable and are attachable and removable from the distal ferrule and the optical fiber and wherein the body and the tip cover the distal ferrule and the optical fiber when attached.
